(19) United States
(12) Patent Application Publication
(10) Pub. No.: US 2007/0259042 A1
(43) Pub. Date: Nov. 8, 2007

(54) Cyclopropanated Aromachemicals

(75) Inventor: Luca Turin, London (GB)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: Flexitral, Inc., Chantilly, VA (US)

(21) Appl. No.: 11/695,485

(22) Filed: Apr. 2, 2007

Related U.S. Application Data
(60) Provisional application No. 60/788,063, filed on Apr. 3, 2006.
(60) Provisional application No. 60/788,064, filed on Apr. 3, 2006.
(60) Provisional application No. 60/827,913, filed on Oct. 3, 2006.

(51) Int. Cl. A61K 8/37; C07C 27/10; C07D 307/02

(52) U.S. Cl. 424/484; 424/48; 424/65; 424/70.1; 424/76.1; 426/531; 426/534; 426/590; 426/87; 510/107; 510/119; 510/130; 510/276; 510/515; 512/25; 514/724; 549/430; 549/523; 560/1; 560/19; 568/420; 568/700; 568/704

(57) ABSTRACT

A compound of formula (I): 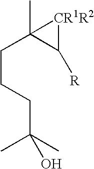
wherein R is —CH₂OH, —C(O)H, —C(OR³)₂H, —C(OR⁴)(OH)H, —CH═NC₆H₄C(O)OR⁵ or —CH═NR⁶;
R¹ and R² are each independently H or CH₃;
R³ and R⁴ are each independently H or a straight or branched aliphatic group having from 1 to 12 carbon atoms, or each R³ is covalently linked to the other R³ to form a cyclic acetal having from 1 to 4 carbon atoms;
R⁵ is H or a straight or branched aliphatic group, or a cyclic, heterocyclic or aromatic group having from 1 to 12 carbon atoms; and
R⁶ has at least 10 carbon atoms and is an aliphatic group or an aromatic group. Mixtures of these compounds, methods for their preparation, their use as perfume materials for application to a variety of substrates and their use in flavouring and in articles of manufacture is also provided.

### CROSS REFERENCE TO RELATED APPLICATIONS

The present application claims priority to Provisional Application Nos. 60/788,063, filed Apr. 3, 2006, 60/788,064, filed Apr. 3, 2006 and 60/827,913 filed Oct. 3, 2006, the contents of each are hereby incorporated by reference in their entireties.

### FIELD OF INVENTION

The present invention relates generally to the field of flavours and fragrances. More particularly, the present invention relates to new flavour and fragrance compounds. These compounds find utility in any and all applications requiring flavours and fragrances. The invention also provides mixtures of these compounds, methods for their preparation and their use as perfume materials for application in a variety of substrates and their use in flavouring and articles of manufacture and compositions including the compounds.

### BACKGROUND OF INVENTION

There are a large number and variety of known flavours and fragrances used as ingredients in perfumes and in a varied range of other products. However, many aromachemicals include double bonds and/or other reactive groups that are potentially susceptible to reaction and may result in a limited useful lifetime. Further, many essential oil fragrances have recently been determined to cause allergic reactions, and it is becoming increasingly difficult to bring products containing such fragrances to market. There is also a demand for new flavours and fragrances that have novel or improved fragrance and/or flavour profiles and/or other properties that make them particularly useful for use as fragrances and/or flavours.

The listing or discussion of a prior-published document in this specification should not necessarily be taken as an acknowledgement that the document is part of the state of the art or common general knowledge.

It is an object of the invention to provide derivatives of the conventional aromachemical compounds. It is also an object of the invention to provide a method for producing these derivatives.

The present invention provides compounds of formula (I): 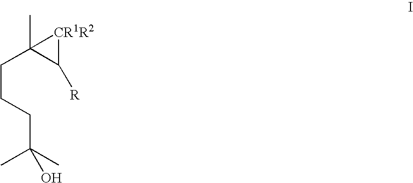
wherein R is —CH₂OH, —C(O)H, —C(OR³)₂H (acetal), —C(OR⁴)(OH)H (hemi-acetal), —CH═NC₆H₄C(O)OR⁵ or —CH═NR⁶, R¹ and R² are each independently H or CH₃, R³ and R⁴ are each independently H or a straight or branched aliphatic group having from 1 to 12 carbon atoms, or each R³ is covalently linked to the other R³ to form a cyclic acetal (ketal) having from 1 to 6 carbon atoms, R⁵ is H or a straight or branched aliphatic group, or a cyclic, heterocyclic or aromatic group having from 1 to 12 carbon atoms, R⁶ has at least 10 carbon atoms and is an aliphatic group or an aromatic group.

R³ and R⁴ preferably have from 1 to 6 carbon atoms, for example 2, 3, 4 or 5 carbon atoms. Preferably R³ and R⁴ are straight chain or branched alkyl groups such as methyl, ethyl, propyl (e.g. n- or i-propyl) or butyl, (e.g. n-, i- or t-butyl). When each R³ is covalently linked to the other R³ to form a cyclic acetal, the cyclic acetal preferably has from 1 to 4 carbon atoms, e.g. 2 or 3 carbon atoms, for example CH₂CH₂ or CH₂CH₂CH₂.

R⁵ preferably has from 1 to 8 carbon atoms and is a straight chain or branched alkyl group such as methyl, ethyl, propyl (e.g. n- or i-propyl) or butyl, (e.g. n-, i- or t-butyl) or an aromatic group.

R⁶ has at least 10 carbon atoms and is preferably alkyl, alkenyl or alkoxy. Preferably R⁶ has from 12 to 25 carbon atoms, for example 18 carbon atoms. The alkyl, alkenyl or alkoxy group may be straight chained or branched. An illustrative example of R⁶ is —(CH₂)₈CH═CH(CH₂)₇CH₃.

In the compounds of formula (I), R¹ and R² may be the same or different. In other words, both R¹ and R² may be H or one of R¹ and R² may be H and the other one may be methyl or R¹ and R² may both be methyl. Preferably, both R¹ and R² represent H.

The compounds of formula (I) are referred to hereinafter as “the compounds of the invention”.

The compounds of the invention typically have one or more of the following improved physical and/or chemical properties relative to the parent compounds such as geraniol and nerol (see below) on which they are based. For example, the compounds of the invention may have increased stability to high or low pH, and/or improved half-life, and/or lower likelihood of causing allergic reactions, and/or increased odour intensity.

Examples of compounds of formula (I) include: 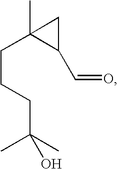
including the isomers:

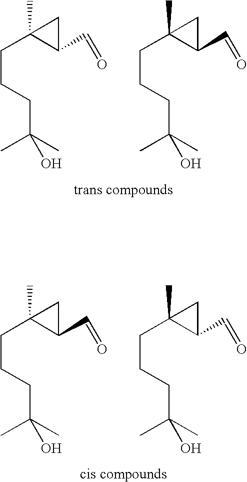

Further examples of compounds of formula (I) include: 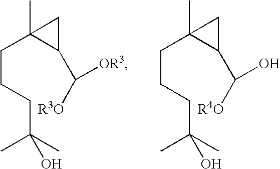
wherein R³ and R⁴ are methyl, ethyl or butyl, for example t-butyl, 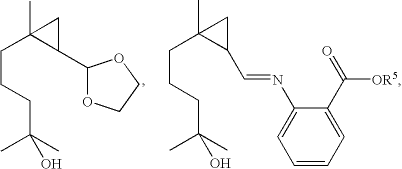
wherein R⁵ is methyl, ethyl or butyl, for example t-butyl or an aromatic group, and

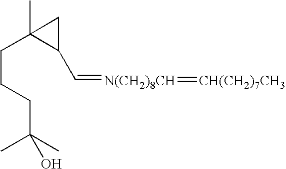

In all of these illustrative compounds one or both of R₁ and R₂ may be methyl rather than H.

Compounds of the invention containing the group R⁶ are preferably liquids of relatively low viscosity at room temperature. For example, the compositions of the present invention containing the group R⁶ are preferably liquid at room temperature (15 to 25° C.) and have a viscosity below about 250 cP, preferably below about 200 cP, when measured at about 20° C. Typically, the preferred compounds of the invention have viscosities below about 100 cP, for example 20 to 100 cP or 50 to 80 cP when measured at about 20° C. The viscosity can be measured using any suitable method known in the art.

Compounds of the invention may contain double bonds and may thus exist as E (entgegen) and Z (zusammen) geometric isomers about each individual double bond. All such isomers and mixtures thereof are included within the scope of the invention.

Compounds of the invention may exhibit tautomerism. All tautomeric forms and mixtures thereof are included within the scope of the invention.

Compounds of the invention may contain one or more asymmetric carbon atoms and may therefore exhibit optical and/or diastereoisomerism. Diastereoisomers may be separated using conventional techniques, e.g. chromatography or fractional crystallisation. The compounds of the invention may be used as a racemic mixture of stereoisomer or may be separated into individual isomers which may then be used separately are in pre-selected ratios. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst, all under conditions known to the skilled person. All stereoisomers and mixtures thereof are included within the scope of the invention.

A racemic mixture of the cis compounds having the formula: 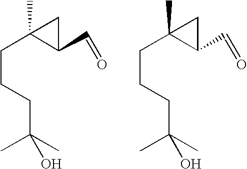
has a lily-of-the-valley, fresh/floral odour. It is believed that one of the many possible uses of these compounds is as a replacement for hydroxycitronellal.

These compounds have a fragrance that is similar to that of hydroxycitronellal but has a significantly more intense odour.

The present invention also provides a process for producing the compounds of formula (I).

The compounds of formula (I) in which R is —C(O)H can be obtained using the following method. It will be appreciated, however, that there are other ways in which the conversions described below can be carried out and that it would not necessarily be necessary to use a reaction scheme that produced each of the intermediates produced in the reaction schemes described below.

Step One

Cyclopropanation of geraniol ((2E)-3,7-dimethyl-2,6-Octadien-1-ol) and/or nerol ((2Z)-3,7-dimethyl-,2,6-Octadien-1-ol) to produce a compound of formula (II):

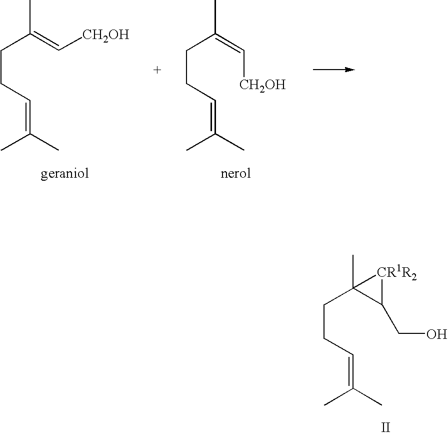

Any suitable method of cyclopropanation known in the art may be used. Suitable methods include carbenoid reactions such the Simmons-Smith cyclopropane synthesis (see for example Vogel's textbook of Practical Organic Chemistry 5^{th} Edition (1989) pp 1106-1108 or Solomon's Organic Chemistry 4^{th} Edition pp 346 and 347, published by John Wiley and Sons). By selection of both suitable reagent and/or conditions (see, for example, Stephenson, PhD thesis, University of Pittsburgh, 2004), the monocyclopropanation reaction using the Simmons-Smith synthesis can be directed to the 2,3-cyclopropanated product (formula II).

Alternatively, the compound of formula (II) shown above can be synthesized by subjecting geraniol and/or nerol to the haloform reaction to produce the dichloro or dibromo cyclopropyl derivative followed by dehalogenation with, e.g., lithium to provide the desired product.

The Friedrichs reaction may also be used to prepare the compound of formula II (see, for example, Friedrich & Lewis, J. Org. Chem., 1990, 55, 2491-2494). In this reaction, acetyl chloride is used to accelerate the cyclopropanation of an alkene with a 1,1,-dibromo or 1,1-diiodo alkyl such as dibromomethane or diiodomethane using zinc dust and copper (I) in ether.

Of the above methods of cyclopropanation, the Friedrichs reaction is currently preferred for preparing the compounds of formula (II) from geraniol/nerol.

The compound of formula (II) has the following stereoeisomers:

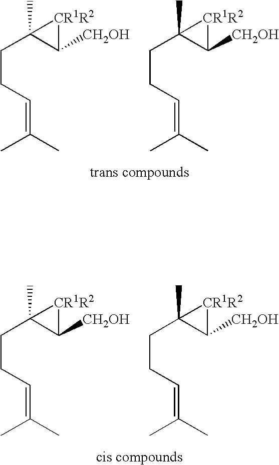

The individual starting materials, geraniol and nerol may be cyclopropanated separately in order to produce the trans- (from geraniol) or cis- (from nerol) compounds of formula (II) as required. Alternatively, a mixture of geraniol and nerol may be cyclopropanated. It is believed that the relative arrangement of the groups is geraniol and nerol is maintained during the cyclopropanation reaction. Thus the use of geraniol alone will typically produce the trans-compounds (as in geraniol) only and the use of nerol alone will typically produce the cis-compounds (as in nerol) only. If a mixture of geraniol and nerol is used, the cyclopropanated produce will contain both the trans- and cis-compounds approximately in the proportions in which the starting material contained geraniol and nerol. When a product containing a mixture of the trans- and cis-compounds is produced, the product may be used for further reaction as a mixture or may be separated into the trans- and cis-compounds by any suitable method known in the art.

Step 2

Epoxidation of the compound of formula (II), for example with m-chloroperbenzoic acid, to produce an oxirane derivative (III). However, any suitable method known in the art for the epoxidation of alkenes may be used (see, for example March, “Advanced Organic Chemistry: Reactions, Mechanisms, and Structure”, 4^{th} Edition, John Wiley & Sons 1992, pages 826 to 829). 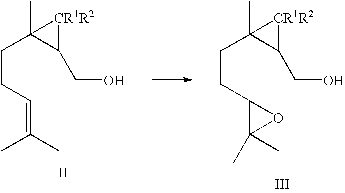
Step 3

Ring opening of the epoxide moiety of the compound of formula (III) by reduction to produce a compound of formula (IV) (the corresponding alcohol). This reaction may be carried out by any suitable method known in the art (see, for example March, “Advanced Organic Chemistry: Reactions, Mechanisms, and Structure”, 4^{th} Edition, John Wiley & Sons 1992, pages 443 and 444) for example by using lithium aluminium hydride.

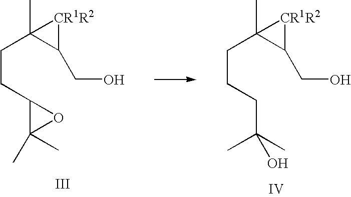

Of course, the compound of formula (IV) is also a compound of formula (I) wherein R is —CH₂OH.

Alternatively, steps 2 and 3 can be replaced by a step in which an addition reaction of water with the compound of formula (II) is carried out to produce the compound of formula (IV). Such an addition reaction may be conducted in the presence of a cation exchanger, as described in U.S. Pat. No. 4,200,766.

Step 4

Oxidation of the compound of formula (IV) to produce the compound of formula (I) in which R is —C(O)H (summarised in the reaction scheme below). This may be achieved using any suitable method known in the art for the oxidation of an alcohol to form an aldehyde (for example as described in March “Advanced Organic Chemistry Reactions, Mechanisms, and Structure”, 4^{th} Edition, John Wiley & Sons 1992, pages 1167 to 1171), for example by using pyridinium dichromate in dichloromethane.

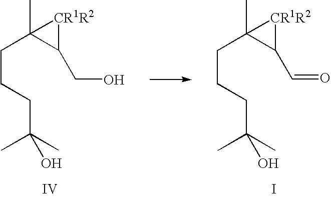

Thus, the cis-compounds 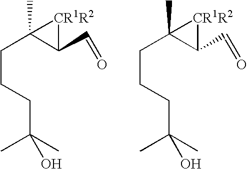
can be produced using the reaction scheme described above using nerol as the initial starting material. The trans-compounds can be produced using the reaction scheme described above using geraniol. Alternatively, a mixture of nerol and geraniol may be used. If a mixture of nerol and geraniol is used the cis- and trans-isomers that are formed may be separated using methods well known in the art at any appropriate stage of the reaction process. For example the isomers of the products of formula (II) or formula (III) or formula (IV) or formula (I) may be separated.

Alternatively, the compounds of the invention may be produced via the monocyclopropanation of geranial and/or neral (the mixture of geranial and neral being known as citral), as illustrated below.

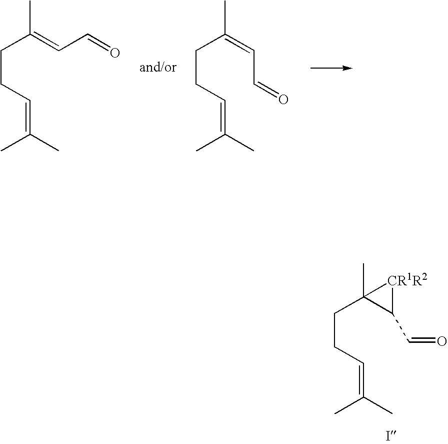

Any suitable cyclopropanation method known in the art may be used to produce the monocyclopropanated aldehydes from geranial, neral or citral, as described above in relation to the cyclopropanation of geraniol and/or nerol.

One method which is suitable for preparing the monocyclopropanated aldehydes is by reaction of geranial and/or neral (or citral) with a suitable sulfoxonium ylide reagent. The use of sulfoxoium ylides in cyclopropanating reactions is described in for example March, “Advanced Organic Chemistry: Reactions, Mechanisms and Structure”, fourth edition (1992), John Wiley & Sons, Inc, page 872.

Suitable sulfoxonium ylides include

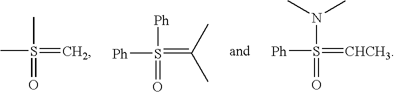

Thus, the monocyclopropanated aldehydes may be prepared by reaction of geranial and/or neral (or citral) as illustrated below.

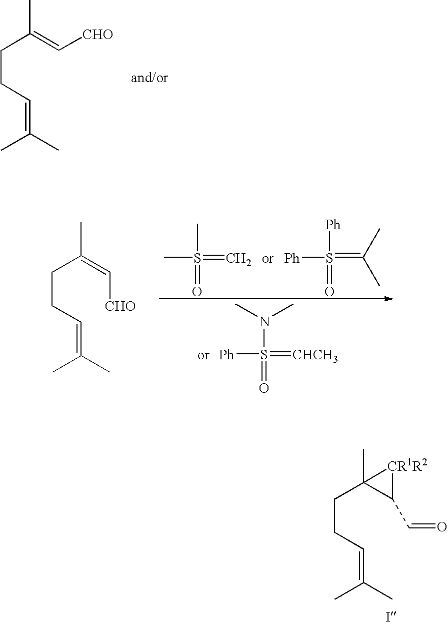

Dimethyloxosulfonium methylide can be obtained by deprotonation of trimethylsulfoxonium iodide with a base such as sodium hydride in any suitable solvent such as DMSO or DMF, preferably under an inert atmosphere (e.g. nitrogen or argon). Corresponding methods can be used to produce the other ylides.

The use of sulfoxoium ylides in cyclopropanating reactions is particularly suitable for the cyclopropanation of conjugated double bonds.

The trans-compounds of the invention may be prepared starting from geraniol (using the Friedrichs reaction for the cyclopropanation step) or starting from geranial (using the reaction with a sulfoxonium ylide).

Similarly, the cis-compounds of the invention may be prepared starting from nerol (using the Friedrichs reaction for the cyclopropanation) or starting from neral (using the reaction with a sulfoxonium ylide).

It will be appreciated that if the aldehyde (geranial and/or neral or citral) is used as the starting material the step of cyclopropanating the aldehyde replaces steps 1 and 4 in the reaction scheme above. The epoxidation and ring opening steps 2 and 3 described above can be used to produce the compounds of the invention starting from the aldehyde. In other words, the step of cyclopropanating the aldehyde is followed by steps 2 and 3 set out above to produce the compounds of the invention.

The compounds of formulae (I) in which R is —C(OR³)₂H or —C(OR⁴)(OH)H can be obtained by known methods for the addition of alcohols to aldehydes, such as treating the compound of formula (I) when R is —C(O)H with an alcohol of formula R³OH or R⁴OH (for example as described in March “Advanced Organic Chemistry: Reactions, Mechanisms, and Structure”, 4^{th} Edition, John Wiley & Sons 1992, pages 889 to 891).

Representative compounds of formulae (I) in which R is —C(OR³)₂H or —C(OR⁴)(OH)H include those in which R³ or R⁴ is methyl, ethyl, propyl (e.g. n- or i-propyl) or butyl (e.g. n-, i- or t-butyl). When R is —C(OR³)₂H, each R³ may also be covalently linked to the other R³ to form a cyclic acetal, preferably containing from 1 to 4 carbon atoms. Such cyclic acetals can be obtained by reacting a compound of formula (I) when R is —C(O)H with HO(CH₂)ₙOH (where n=1 to 4), in the presence of an acid.

The compounds of formula (I) in which R is —CH═NC₆H₄C(O)OR⁵ can be obtained by the reaction of a compound of formula (I) in which R is —C(O)H with a primary amine of formula H₂C₆H₄C(O)OR⁵.

The compounds of formula (I) in which R is —CH═NR⁶ can be obtained by the reaction of a compound of formula (I) in which R is —C(O)H with a primary amine of formula H₂NR⁶.

The reaction of primary amines to form the imines of formula (I) in which R is —CH═NC₆H₄C(O)OR⁵ or —CH═NR⁶ may be carried out by any suitable method known in the art, for example as described in March “Advanced Organic Chemistry: Reactions, Mechanisms, and Structure”, 4^{th} Edition, John Wiley & Sons 1992, pages 896 and 897.

The amine compounds of formula H₂NR⁶ typically have an Odor Intensity Index of less than that of a 1% solution of methylanthranilate in dipropylene glycol, and a Dry Surface Odor Index of more than 5.

To measure the Odor Intensity Index, it is meant that the pure chemical is diluted at 1% in dipropylene glycol, an odor-free solvent used in perfumery. This percentage dilution is more representative of usage levels. Smelling strips or so called “blotters”, are dipped and presented to the expert panelist for evaluation. Expert panelists are assessors trained for at least six months in odor grading and whose gradings are checked for accuracy and reproducibility versus a reference on an on-going basis. For each amine compound, the panelist is presented with two blotters: one reference methylanthranilate) and the sample. The panelist is asked to rank both smelling strips on the 0 to 5 odor intensity scale, 0 being no odor detected and 5 being very strong odor present.

Suitable amines of formula H₂NR⁶ are preferably non-fragrant, odorless, non-volatile amines having a relatively low vapor pressure and high molecular weight, i.e. aromatic or aliphatic amines containing more than about 10 carbon atoms. Preferably the amines have a molecular weight of at least 150 daltons.

Suitable amines of formula H₂NR⁶ include odourless, low vapour pressure aliphatic or aromatic amines containing at least one free, unmodified primary amino group. Any suitable alkyl, alkenyl or alkoxy, branched or straight chain amine having a total of at least 10 carbon atoms that is relatively odourless and forms a relatively insoluble derivative with the aromachemical that has a relatively low viscosity may be employed. Suitable amines include but are not limited to n-dodecylamine, n-tetradecylamine, n-hexadecylamine, n-octadecylamine, oleylamine, cocoalkylamines, soyaalkylamines, tallowalkylamines, hydrogenated tallowalkylamines, branched isomers and/or derivatives thereof and mixtures thereof.

As used herein, the term “primary amine” is meant to include a component that carries at least one primary amine and/or amide function.

The present invention provides for the use of the compounds of the invention and mixtures thereof as a flavor and/or fragrance.

The present invention also provides compositions, products, preparations or articles containing a compound or mixture of compounds of the invention as described above.

The present invention also provides methods to confer, improve, enhance or modify the taste or flavor property of a composition, product, preparation or article which comprises adding thereto a flavor effective amount of a composition or mixture of compounds of the invention as described above.

A method to confer, improve, enhance or modify the aroma, fragrance or odor characteristics of compositions, products, preparations or articles which comprises adding thereto an aroma, fragrance or odor effective amount of a composition or mixture of compounds of the invention as described above is also provided.

The compounds of the invention can be included in virtually any article of manufacture that can include fragrance or flavorant compounds. Examples include hypochlorite (bleach) compositions, detergents, flavorings and fragrances, beverages, including alcoholic beverages, and the like. The compounds of the invention can be used in applications like soaps, shampoos, denture cleanser tablets, body deodorants and antiperspirants, solid or liquid detergents for treating textiles, fabric softeners, detergent compositions and/or all-purpose cleaners for cleaning dishes or various surfaces, for both household and industrial use. Of course, the use of the compounds is not limited to the above-mentioned products, as they be used in other current uses in perfumery, namely the perfuming of soaps and shower gels, hygiene or hair-care products, as well as of body deodorants, air fresheners and cosmetic preparations, and even in fine perfumery, namely in perfumes and colognes.

The compounds of the invention also find utility in foods, flavorings, beverages such as beer and soda, denture cleansers (tablets), flavored orally-delivered products such as lozenges, candies, chewing gums, matrices, pharmaceuticals and the like. These uses are described in more detail below.

The compounds of the invention can be used as perfuming ingredients, as single compounds or as mixtures thereof. The compounds can be used in their pure state or as mixtures, without added components. The olfactive characteristics of the individual compounds are also present in mixtures thereof, and mixtures of these compounds can be used as perfuming ingredients. This may be particularly advantageous where separation and/or purification steps can be avoided by using compound mixtures.

In all of the above applications, the compounds of the invention can be used alone, in admixture with each other, or in admixture with other perfuming ingredients, solvents or adjuvants of current use in the art. The nature and the variety of these co-ingredients do not require a more detailed description here, which, moreover, would not be exhaustive, and the person skilled in the art will be able to choose the latter through their general knowledge and as a function of the nature of the product to be perfumed and of the desired olfactive effect.

These perfuming ingredients typically belong to chemical classes as varied as alcohols, aldehydes, ketones, esters, ethers, acetates, nitrites, terpene hydrocarbons, sulfur- and nitrogen containing heterocyclic compounds, as well as essential oils of natural or synthetic origin. A large number of these ingredients described in reference textbooks such as the book of S. Arctander, Perfume and Flavor Chemicals, 1969, Montclair, N.J., USA, the contents of which are hereby incorporated by reference in its entirety, or its more recent versions, or in other works of similar nature.

The proportions in which the compounds of the invention can be incorporated in the various products vary within a large range of values. These values depend on the nature of the article or product that one desires to perfume and the odor effect searched for, as well as on the nature of the co-ingredients in a given composition when the compounds are used in admixture with perfuming co-ingredients, solvents or adjuvants of current use in the art.

As an example, the compounds of the invention are typically present at concentrations between about 0.01 and about 30%, or even more, by weight of these compounds relative to the weight of the composition, product or article in which they are incorporated. It will be appreciated that the amount by weight of a compound of the invention in a particular composition or product will depend on the nature of the composition. For example, a washing powder will typically contain less than 1% by weight of a compound of the invention while a fine fragrance may contain more than 20% by weight of a compound of the invention.

The compounds may be used in detergents such as those containing bleaching agents and activators such as, for example, tetraacetylethylenediamine (TAED), hypohalites, in particular hypochlorite, peroxygenated bleaching agents such as, for example, perborates, etc. The compounds can also be used in body deodorants and antiperspirants, for example, those containing aluminum salts. These aspects are described in more detail below.

In addition to the compounds of the invention, the compositions described herein may include a detersive surfactant and optionally, one or more additional detergent ingredients, including materials for assisting or enhancing cleaning performance, treatment of the substrate to be cleaned, or to modify the aesthetics of the detergent composition (e.g. perfumes, colorants, dyes, etc.). Non-limiting examples of synthetic detersive surfactants useful herein typically at levels from about 0.5% to about 90%, by weight, include the conventional C₁₋₁₈ alkyl benzene sulfonates (“LAS”) and primary, branch-chain and random C₁₀₋₂₀ alkyl sulfates (“AS”), and the like. Preferred compositions incorporating only synthetic detergents have a detergent level of from about 0.5% to 50%. Compositions containing soap preferably comprise from about 10% to about 90% soap.

The compositions described herein can contain other ingredients such as enzymes, bleaches, fabric softening agents, dye transfer inhibitors, suds suppressors, and chelating agents, all well known within the art.

The compounds of the invention can be incorporated into beverages and impart various flavorings to the beverages. The beverage composition can be a cola beverage composition, and can also be coffee, tea, dairy beverage, fruit juice drink, orange drink, lemon-lime drink, beer, malt beverages, or other flavored beverage. The beverages can be in liquid or powdered form. The beverage compositions can also include one or more flavoring agents; artificial colorants; vitamin additives; preservatives; caffeine additives; water; acidulants; thickeners; buffering agents; emulsifiers; and/or fruit juice concentrates.

Artificial colorants that may be used include caramel color, yellow 6 and yellow 5. Useful vitamin additives include vitamin B2, vitamin B6, vitamin B12, vitamin C (ascorbic acid), niacin, pantothenic acid, biotin and folic acid. Suitable preservatives include sodium or potassium benzoate. Salts that may be used include sodium, potassium and magnesium chloride. Exemplary emulsifiers are gum arabic and purity gum, and a useful thickener is pectin. Suitable acidulants include citric, phosphoric and malic acid, and potential buffering agents include sodium and potassium citrate.

The beverage may, for example, be a carbonated cola beverage. The pH is generally about 2.8 and the following ingredients can be used to make the syrup for these compositions: Flavor Concentrate, including one or more of the compounds of the invention herein (22.22 ml), 80% Phosphoric Acid (5.55 g), Citric Acid (0.267 g), Caffeine (1.24 g), artificial sweetener, sugar or corn syrup (to taste, depending on the actual sweetener) and Potassium Citrate (4.07 g). The beverage composition can be prepared, for example, by mixing the foregoing syrup with carbonated water in a proportion of 50 ml syrup to 250 ml of carbonated water.

Flavored food and pharmaceutical compositions including one or more of the compounds of the invention can also be prepared. The compounds of the invention can be incorporated into conventional foodstuffs using techniques well known to those of skill in the art. Alternatively, the compounds can be incorporated within polymeric particles, which can, in turn, be dispersed within and/or over a surface of an orally-deliverable matrix material, which is usually a solid or semi-solid substrate. When used in chewable compositions, the compounds of the invention can be released into the orally-deliverable polymeric matrix material as the composition is chewed and held in the mouth, thus prolonging the flavor of the composition. In the case of dried powders and mixes, the flavor can be made available as the product is consumed or be released into the matrix material as the composition is further processed. When two flavors are combined with the polymeric particles, the relative amounts of the additives can be selected to provide simultaneous release and exhaustion of the compounds.

Flavored compositions of the invention may include an orally-deliverable matrix material; a plurality of water insoluble polymeric particles dispersed in the orally-deliverable matrix material, where the polymeric particles individually define networks of internal pores and are non-degradable in the digestive tract; and one or more compounds of the invention entrapped within the internal pore networks. The compounds of the invention are released as the matrix is chewed, dissolved in the mouth, or undergoes further processing selected from the group consisting of liquid addition, dry blending, stirring, mixing, heating, baking, and cooking. The orally-deliverable matrix material can be selected from the group consisting of gums, latex materials, crystallized sugars, amorphous sugars, fondants, nougats, jams, jellies, pastes, powders, dry blends, dehydrated food mixes, baked goods, batters, doughs, tablets, and lozenges.

A flavorless gum base can be combined with a compound or a mixture of compounds of the invention to a desired flavor concentration. In one method for producing such gum based products a blade mixer is heated to about 110° F., the gum base is preheated so that it is softened, and the gum base is then added to the mixer and allowed to mix for approximately 30 seconds. The compound or compounds of the invention are then added to the mixer and mixed for a suitable amount of time. The gum can be then removed from the mixer and rolled to stick thickness on waxed paper while warm.

The compounds of the invention may be incorporated into a system that can release a fragrance in a controlled manner. These include substrates such as air fresheners, laundry detergents, fabric softeners, deodorants, lotions, and other household items. The fragrances are generally one or more derivatives of essential oils as described herein, each present in different quantities. U.S. Pat. No. 4,587,129, the contents of which are hereby incorporated by reference in their entirety, describes a method for preparing gel articles that contain up to 90% by weight of fragrance or perfume oils. The gels are prepared from a polymer having a hydroxy (lower alkoxy) 2-alkeneoate, a hydroxy (lower alkoxy) lower alkyl 2-alkeneoate, or a hydroxy poly (lower alkoxy) lower alkyl 2-alkeneoate and a polyethylenically unsaturated crosslinking agent. These materials have continuous slow release properties, i.e. they release the fragrance component continuously over a long period of time. Advantageously, all or a portion of those derivatives that include an aldehyde group can be modified to include an acetal group, which can cause the formulations to release fragrance over a period of time as the acetal hydrolyzes to form the aldehyde compound.

The present invention is illustrated by the following non-limiting example.

### EXAMPLE

### Synthesis of 2-(4-hydroxy-4-methylpentyl)-2-methylcyclopropane carbaldehyde

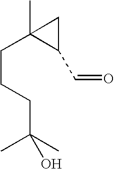

### Step 1

### Synthesis of (2-methyl-2-(4-methylpent-3-enyl)cyclopropyl)methanol

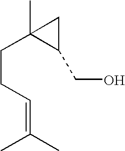

Dibromomethane (2.25 ml, 32.4 mmol) and acetyl chloride (0.2 ml, 3.2 mmol) were added to a suspension of zinc dust (8.47 g, 0.13 mol) and copper chloride (1.27 g, 12.9 mmol) in diethyl ether (30 ml) at room temperature. The mixture was stirred 10 minutes before Geraniol/Nerol (5 g, 32.4 mmol), and dibromothane (2.25 ml, 32.4 mmol) were added. The reaction mixture was stirred for 90 minutes, and then poured into an ammonium chloride solution at 0° C. The aqueous phase was extracted with ethyl acetate (3×100 ml). The aqueous extract were dried over magnesium sulphate and evaporated. Purification by silica gel chromatography with a gradient of 2-5% ethyl acetate/hexane provided the desired compound as colourless oil (3.08 g, 18.3 mmol) in 57% yield.

This reaction was scaled-up and improved as follows. Dibromomethane (13.5 ml, 0.19 mol) and acetyl chloride (1.2 ml, 19.2 mmol) were added to a suspension of zinc dust (38 g, 0.58 mol) and copper chloride (5.74 g, 0.06 mol) in diethyl ether (200 ml) at room temperature. The mixture was stirred 10 minutes before geraniol/nerol (30 g, 0.19 mol), and dibromethane (2.25 ml, 32.4 mmol) were added dropwise. The reaction was kept below 20° C. during and after the addition. The reaction mixture was stirred overnight, and then poured into an ammonium chloride solution at 0° C. The aqueous phase was extracted with ethyl acetate (3×500 ml). The organic phases were dried over magnesium sulphate and evaporated. Purification by silica gel chromatography with a gradient of 2-5% ethyl acetate/hexane provided the desired compound as colourless oil (25.3 g, 0.15 mol) in 80% yield.

### Step 2

### Synthesis of (2-methyl-2-(2-(3,3-dimethyloxiran-2-yl)ethyl)cyclopropyl)methanol

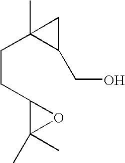 m-chloroperbenzoic acid (77%) (1.66 g, 7.37 mmol) was added portion wise to a solution of (2-methyl-2-(4-methylpent-3-enyl)cyclopropyl)methanol (1 g, 5.9 mmol) in dichloromethane (30 ml) at 0° C. The reaction mixture was stirred for 3 hours at 0° C. The solution washed with aqueous solutions of sodium sulfite and sodium carbonate, and dried over magnesium sulfate. Purification by silica gel chromatography with a gradient of 0-10% ethyl acetate/dichloromethane provided the desired compound as colourless oil (601 mg, 3.2 mmol) in 55% yield.

### Step 3

### Synthesis of 5-(2-(hydroxymethyl)-1-methylcyclopropyl)-2-methylpentan-2-ol

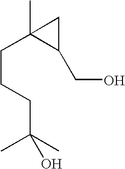

A solution of (2-methyl-2-(2-(3,3-dimethyloxiran-2-yl)ethyl)cyclopropyl)methanol (250 mg, 1.35 mmol) in diethyl ether (5 ml) was added to a suspension of lithium aluminium hydride (6.20 mg, 1.63 mmol) in diethyl ether (10 ml). The reaction mixture was stirred at room temperature for 2 hours. Ethyl acetate (10 ml) was slowly added to the reaction followed by water (20 ml). The aqueous phase was extracted three times with ethyl acetate (20 ml). The combined organic phases were dried over magnesium sulfate and evaporated under vacuum to afford the desired product (240 mg, 1.29 mmol) in 95% yield.

### Step 4

### Synthesis of 2-(4-hydroxy-4-methylpentyl)-2-methylcyclopropane carbaldehyde

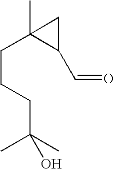

A solution of 25-(2-(hydroxymethyl)-1-methylcyclopropyl)-2-methylpentan-2-ol (900 mg, 4.8 mmol) in dichloromethane (20 ml) was added to a solution of pyridinium dichromate (2.37 g, 6.3 mmol) in dichloromethane (50 ml) at 0° C. The reaction mixture was stirred at 0° C. for 30 minutes then at room temperature for 3 hours. The mixture was diluted in diethyl ether (60 ml) and filtered through celite. After evaporation of the solvent the crude product was purified by silica gel chromatography eluting with dichloromethane provided the desired compound as colourless oil (750 mg, 4.08 mmol) in 85% yield.

Having hereby disclosed the subject matter of the present invention, it should be apparent that many modifications, substitutions, and variations of the present invention are possible in light thereof. It is to be understood that the present invention can be practiced other than as specifically described. Such modifications, substitutions and variations are intended to be within the scope of the present application.

1. A compound of formula (I): 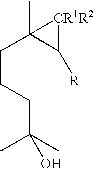
wherein R is —CH₂OH, —C(O)H, —C(OR³)₂H, —C(OR⁴)(OH)H, —CH═NC₆H₄C(O)OR⁵ or —CH═NR⁶;
R¹ and R² are each independently H or CH₃;
R³ and R⁴ are each independently H or a straight or branched aliphatic group having from 1 to 12 carbon atoms, or each R³ is covalently linked to the other R³ to form a cyclic acetal having from 1 to 4 carbon atoms;
R⁵ is H or a straight or branched aliphatic group, or a cyclic, heterocyclic or aromatic group having from 1 to 12 carbon atoms; and
R⁶ has at least 10 carbon atoms and is an aliphatic group or an aromatic group.

2. A compound of claim 1 wherein R is —C(O)H.

3. A compound of claim 2 having the following formulae:

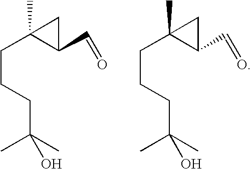

4. A compound of claim 1 wherein R is —C(OR³)₂H having the following formula: 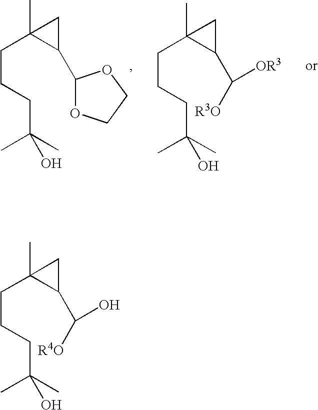
wherein R³ and R⁴ are methyl, ethyl or butyl.

5. A compound of claim 1 wherein R is —CH═NC₆H₄C(O)OR⁵ having the following formula:

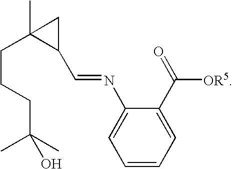

6. A compound of claim 1 wherein R is —CH═NR⁶ having the following formula:

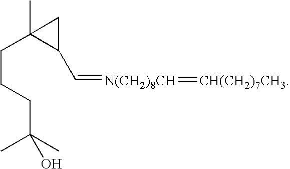

7. A compound of claim 1 wherein R¹ and R² represent H.

8. A method of producing a compound of formula (I) as defined in claim 1 comprising a step in which geraniol and/or nerol is cyclopropanated to produce a compound of formula (II):

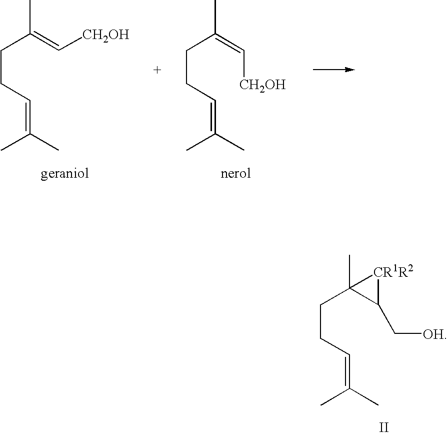

9. A method according to claim 8 wherein the cyclopropanation of geraniol and/or nerol is carried out using a Friedrichs reaction.

10. A method of producing a compound of formula (I) as defined in claim 1 comprising epoxidizing the compound of formula (II) to produce a compound of formula (III):

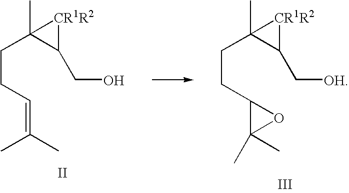

11. A method of producing a compound of formula (I) as defined in claim 1 comprising converting a compound of formula (III) to a compound of formula (IV):

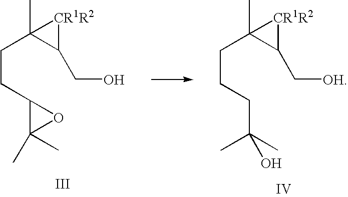

12. A method of producing a compound of formula (I) as defined in claim 1 comprising oxidizing a compound of formula (IV) to produce a compound of formula (I) wherein R is —C(O)H:

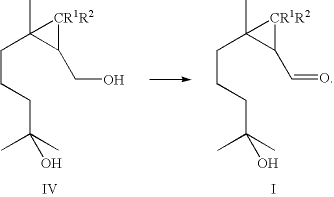

13. A method of producing a compound according to claim 1 in which R is —C(OR³)₂H or —C(OR⁴)(OH)H comprising reacting a compound of formula (I) wherein R is —C(O)H with R³OH or R⁴OH to produce a compound of formula (I) wherein R is —C(OR³)₂H or —C(OR⁴)(OH)H.

14. A method of producing a compound according to claim 1 in which R is —CH═NC₆H₄C(O)OR⁵ or —CH═NR⁶ comprising reacting a compound of formula (I) wherein R is —C(O)H with a primary amine of formula H₂C₆H₄C(O)OR⁵ or H₂NR⁶ to produce a compound of formula (I) wherein R is —CH═NC₆H₄C(O)OR⁵ or —CH═NR⁶.

15. A composition comprising a flavour or fragrance enhancing amount of a compound or mixture of compounds as defined in claim 1.

16. A substrate treated with a compound or mixture of compounds as defined in claim 1.

17. A method for treating a substrate to impart flavorant/fragrance releasing characteristics thereto comprising treating the substrate with a flavour or fragrance enhancing amount of a compound or mixture of compounds as defined in claim 1.

18. A composition, product, preparation or article having aroma, fragrance or odor releasing characteristics containing a compound or mixture of compounds as defined in claim 1 in admixture with at least one other perfuming ingredient, solvent, or adjuvant.

19. A composition, product, preparation or article according to claim 18 in the form of a perfume, fragrance or cologne, a soap, a bath or shower gel, a shampoo or other hair care product, a cosmetic preparation, a body odorant, deodorant or antiperspirant, an air freshener, a liquid or solid fabric detergent or softener, bleach product, disinfectant or an all-purpose household or industrial cleaner.

20. A composition, product, preparation or article according to claim 19, wherein the compound or mixture of compounds is in admixture with at least one bleach ingredient and/or at least one detergent ingredient and/or at least one disinfectant ingredient.

21. A composition, product, preparation or article according to claim 19 in the form of a body odorant, deodorant or antiperspirant wherein the compound or mixture of compounds is in admixture with other body odorant, deodorant or antiperspirant ingredients, solvents and/or adjuvants.

22. A beverage composition comprising a compound or mixture of compounds according to claim 1 and one or more additional beverage ingredients.

23. A flavouring composition, product or article comprising a compound or mixture of compounds according to claim 1 and a solvent, adjuvant and/or another flavouring ingredient.

24. A food composition, product, preparation or article comprising a compound or mixture of compounds according to claim 1 and at least one solvent, adjuvant, and/or another food ingredient.

25. A chewing gum composition, product, preparation or article comprising a compound or mixture of compounds according to claim 1 and at least one solvent, adjuvant, and/or another chewing gum ingredient.

26. A pharmaceutical composition, product, preparation or article comprising a compound or mixture of compounds according to claim 1 and a pharmaceutically active ingredient.

27. An orally-deliverable matrix comprising a compound or mixture of compounds according to claim 1 and at least one matrix material.

28. A method to confer, improve, enhance or modify a taste or flavor property of a composition, product, preparation or article which comprises adding thereto a flavor effective amount of a compound or mixture of compounds as defined in any of claim 1.

29. A method according to claim 28, wherein said composition, product, preparation or article is in the form of a beverage, a flavoring, a food, a chewing gum, a pharmaceutical or an orally deliverable matrix.

30. A method to confer, improve, enhance or modify an aroma, fragrance or odor characteristics of a composition, product, preparation or article which comprises adding thereto an aroma, fragrance or odor effective amount of a compound or mixture of compounds as defined in claim 1.

31. A method according to claim 30 wherein said composition, product, preparation or article is in the form of a perfume, a body odorant, deodorant or antiperspirant, a detergent, a bleach product or a disinfectant.

32. An article of manufacture comprising packaging material and an aroma, odor, fragrance, taste or flavor enhancing agent contained within the packaging material, wherein the agent is effective for the enhancement of the aroma, odor, fragrance, taste or flavor of a composition, preparation, product or article to which it is added, and wherein the packaging material comprises a label which indicates that the agent can be used for enhancing aroma, odor, fragrance, taste or flavor, and wherein the agent is a compound or mixture of compounds as defined in claim 1.
